(19) United States
(12) Patent Application Publication
(10) Pub. No.: US 2012/0143243 A1
(43) Pub. Date: Jun. 7, 2012

(54) CLOSURE DEVICES

(75) Inventors: Jason P. Hill, Brooklyn Park, MN (US); Horng-Ban Lin, Maple Grove, MN (US); James Q. Feng, Maple Grove, MN (US); Scott R. Schewe, Eden Prairie, MN (US); John B. Blix, Osseo, MN (US)

(73) Assignee: Boston Scientific Scimed, Inc., Maple Grove, MN (US)

(21) Appl. No.: 13/310,429

(22) Filed: Dec. 2, 2011

Related U.S. Application Data
(60) Provisional application No. 61/419,588, filed on Dec. 3, 2010.

(51) Int. Cl. A61B 17/08

(52) U.S. Cl. 606/213

(57) ABSTRACT

One or more embodiments of the present disclosure include a closure device, a method of making the closure device and a system that includes the closure device for closing an opening in a body lumen. The closure device includes an intravascular anchor for positioning in the body lumen, a suture coupled to the intravascular anchor, the suture to pass through the opening in the body lumen, a fastener joined to the suture, where the fastener moves longitudinally along the suture, and a plug having a first plug segment and a second plug segment. In one or more embodiments, the second plug segment has a hardness greater than the first plug segment, where the suture passes through the plug with the first plug segment between the intravascular anchor and the second plug segment and the second plug segment between the first plug segment and the fastener.

### PRIORITY INFORMATION

This application claims priority to U.S. Provisional Application No. 61/419,588 filed on Dec. 3, 2010, the specification of which is incorporated herein by reference.

### FIELD OF DISCLOSURE

Embodiments of the present disclosure are directed toward closure devices; more specifically, embodiments are directed toward closure devices for closing an opening in a body lumen.

### BACKGROUND

For a diagnostic and/or interventional catherization procedure, such as a coronary procedure, a small gauge needle is introduced through a patient's skin to a target blood vessel, such as the femoral artery in the region of the patient's groin. The needle forms a puncture, i.e. an arteriotomy, through the blood vessel wall. A guide wire is then introduced through the needle, and the needle withdrawn over the guide wire. An introducer-sheath is next introduced over the guide wire, and the sheath and guide wire may be left in place to provide access during the procedure. Examples of procedures include diagnostic procedures such as angiography, ultrasonic imaging, and the like, and interventional procedures, such as angioplasty, atherectomy, stent placement, laser ablation, graft placement, and the like.

After the procedure is completed, the catheters, guide wire, and introducer-sheath are removed, and it is necessary to close the arteriotomy to provide hemostasis (i.e., stop blood loss) and allow healing.

### SUMMARY

One or more embodiments of the present disclosure include a closure device for closing an opening in a body lumen. The closure device includes an intravascular anchor for positioning in the body lumen, a suture coupled to the intravascular anchor, the suture to pass through the opening in the body lumen, a fastener joined to the suture, where the fastener moves longitudinally along the suture, and a plug having a first plug segment and a second plug segment. In one or more embodiments, the second plug segment has a hardness greater than the first plug segment, where the suture passes through the plug with the first plug segment between the intravascular anchor and the second plug segment and the second plug segment between the first plug segment and the fastener.

For the various embodiments, the first plug segment and the second plug segment forming the plug can have a variety of configurations. For example, in one embodiment the second plug segment can be a composite of constituent materials that include a portion of the first plug segment and a matrix material that surrounds and supports the portion of the first plug segment. In an additional example, the second plug segment can be a composite of constituent materials that include a portion of the first plug segment and a mesh of a reinforcement material that surrounds and supports the portion of the first plug segment. In a further example, the second plug segment can have a hardness that is greater than the first plug segment.

One or more embodiments of the present disclosure include a system for closing an opening in a body lumen. In one or more embodiments, the system includes a sheath and the closure device releasably housed in the sheath, where the closure device is as described herein, and a push member disposed in the sheath, where the push member extends to advance the closure device from the sheath.

One or more embodiments of the present disclosure include a method of making the closure device for closing an opening in a body lumen. In one or more embodiments, the method includes forming a first plug segment of a plug of the closure device, forming a second plug segment of the plug of the closure device, where forming the second plug segment includes making a hardness of the second plug segment greater than the first plug segment, passing a suture through the plug with the first plug segment between an intravascular anchor and the second plug segment, and joining a fastener to the suture with the second plug segment between the first plug segment and the fastener.

The above summary of the present disclosure is not intended to describe each disclosed embodiment or every implementation of the present disclosure. The description that follows more particularly exemplifies illustrative embodiments. In several places throughout the application, guidance is provided through various embodiments and examples, which embodiments and examples can be used in various combinations. In each instance, the recited embodiments and examples serve only in a representative function and should not be interpreted as being the exclusive list of embodiments and/or examples.

### BRIEF DESCRIPTION OF THE FIGURES

FIGS. 1A-1C illustrate a closure device according to an embodiment of the present disclosure.

FIGS. 2A-2C illustrate a closure device according to an embodiment of the present disclosure.

FIGS. 3A-3C illustrate a closure device according to an embodiment of the present disclosure.

FIGS. 4A-4C illustrate a closure device according to an embodiment of the present disclosure.

FIG. 5 illustrates a closure device according to an embodiment of the present disclosure.

FIG. 6 illustrates a system for closing an opening in a body lumen according to an embodiment of the present disclosure.

FIG. 7 illustrates the system for closing an opening in a body lumen disposed within an introducer sheath according to an embodiment of the present disclosure.

The elements illustrated in the Figures are not to scale and are provided to exemplify their relationship within the various embodiments illustrated in the Figures.

### DETAILED DESCRIPTION

Embodiments of the present disclosure provide closure devices for closing an opening in a body lumen. The closure devices include an intravascular anchor, a suture coupled to the intravascular anchor, a fastener joined to the suture and a plug, where the suture passes through the plug. For the various embodiments, the fastener can move longitudinally along the suture to contact and to cause the plug to compress against at least a portion of the intravascular anchor.

For the various embodiments, the strength and robustness of the plug must be sufficient to ensure the compressive axial force applied by and through the fastener can form an effective seal at the arteriotomy. If the plug were to be torn or damaged by the fastener during deployment the result may be poor sealing at the arteriotomy. The embodiments of the present disclosure help to ensure that there is minimal, if any, damage or tearing of the plug during the deployment and compression of the closure device across the arteriotomy. In this way, the compressive axial force of the fastener can be transferred into a controlled compaction (or compression) of the plug for effectively sealing the arteriotomy.

As used herein, “a,” “an,” “the,” “at least one,” and “one or more” are used interchangeably. The term “and/or” means one, one or more, or all of the listed items. The recitations of numerical ranges by endpoints include all numbers subsumed within that range (e.g., 1 to 5 includes 1, 1.5, 2, 2.75, 3, 3.80, 4, 5, etc.).

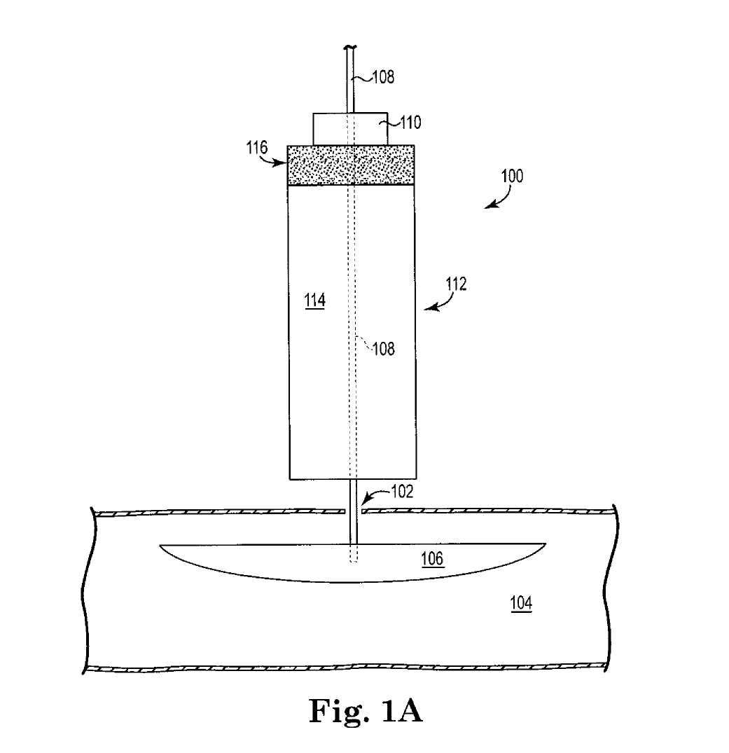

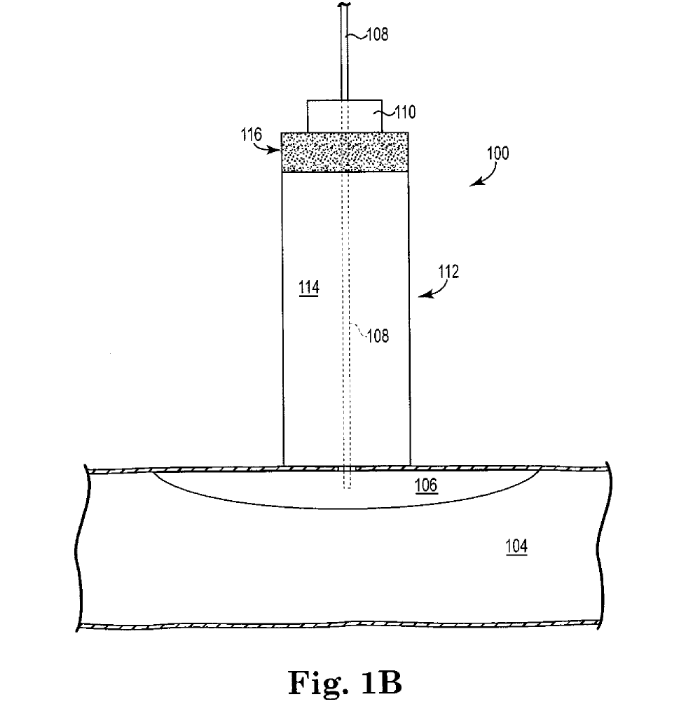

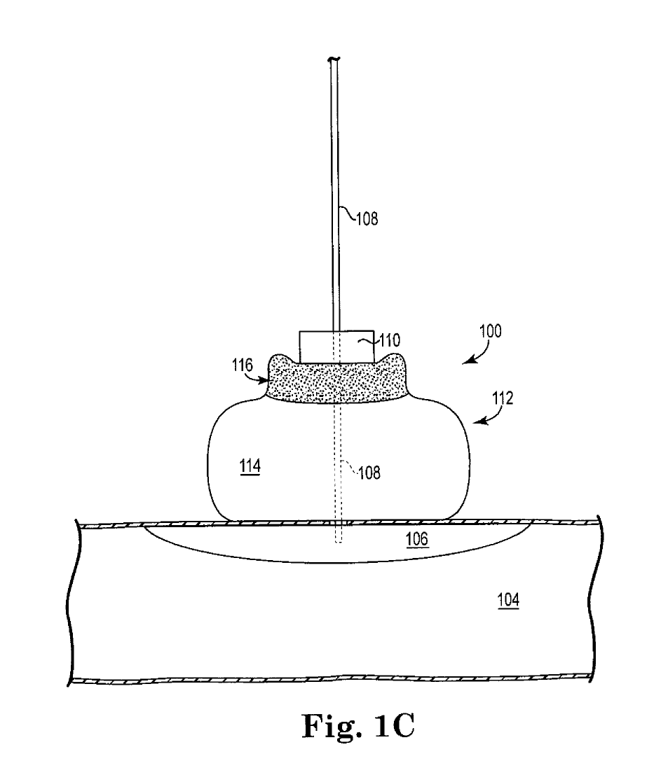

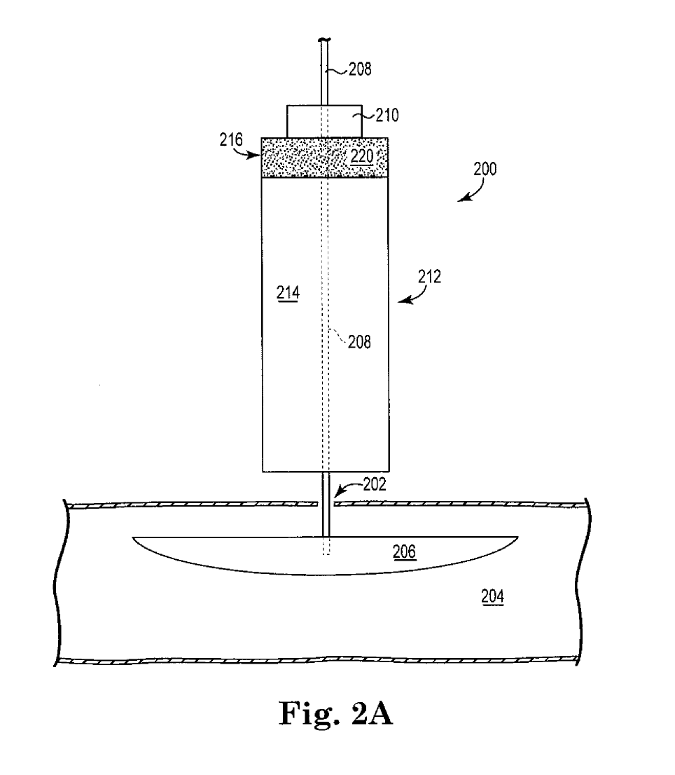

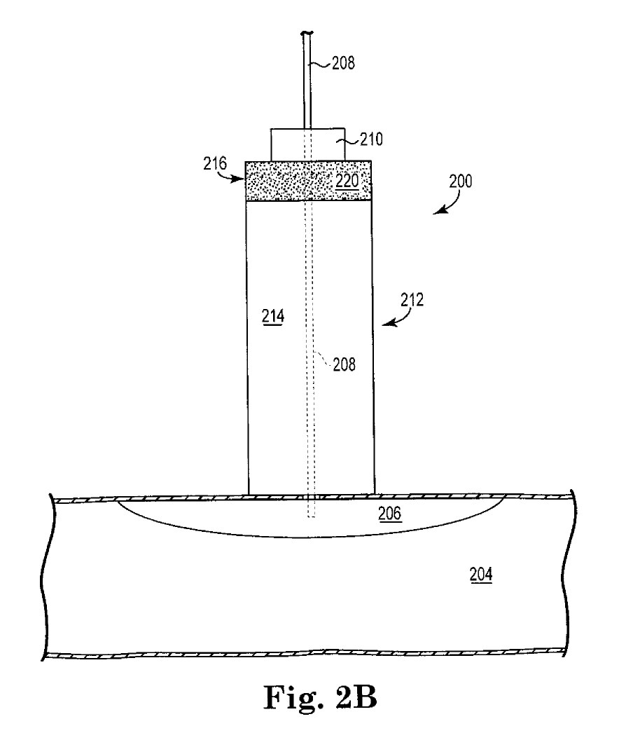

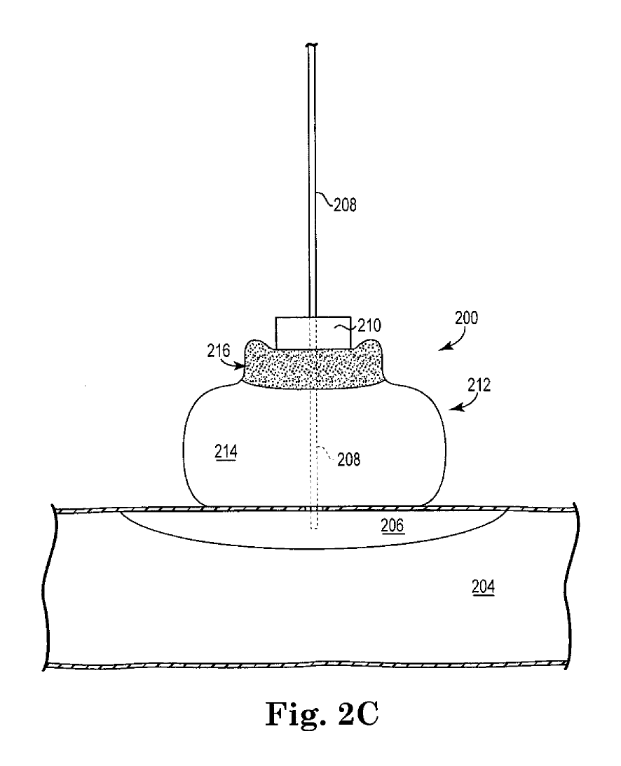

The figures herein follow a numbering convention in which the first digit or digits correspond to the drawing figure number and the remaining digits identify an element in the drawing. Similar elements between different figures may be identified by the use of similar digits. For example, 106 may reference element “06” in FIG. 1, and a similar element may be referenced as 206 in FIG. 2.

Referring to FIGS. 1A-1C there is illustrated an embodiment of a closure device 100 for closing an opening 102 in a body lumen 104. The closure device 100 includes an intravascular anchor 106 for positioning in the body lumen, a suture 108 coupled to the intravascular anchor 106, a fastener 110 joined to the suture 108 and a plug 112. In one or more embodiments, the suture 108 can pass through the opening 102 in the body lumen 104 with the intravascular anchor 106 being positioned within the lumen 104 of the vessel and the plug 112 and fastener 110 being positioned across the opening 102 from the intravascular anchor 106.

In one or more embodiments, the plug 112 has a first plug segment 114 and a second plug segment 116, where the second plug segment 116 has a hardness and/or a strength that is greater than the first plug segment 114. Hardness, as used herein, refers to the property of a material to resist a change of shape (e.g., permanent deformation) when a force is applied to the material. Strength, as used herein, refers to a materials ability to withstand an applied stress without failure. In contrast, a density refers to a materials mass divided by the unit volume. As appreciated, there is no direct or simple correlation between a density of a material and its hardness and/or strength. So, increasing a density of a material does not mean that the hardness and/or strength of the material will also increase. Increases in density can be attributed to packing of the molecules of the material, whereas increases in hardness and/or strength have more to do with the bonding between the atoms of the molecules. So, for example, it is possible to increase the hardness and strength of the second plug segment 116 formed from a collagen foam by altering the cross-link density between the molecules without appreciably affecting its density.

As illustrated in FIGS. 1A-1C, the first plug segment 114 is positioned between the intravascular anchor 106 and the second plug segment 116, and the second plug segment 116 is between the first plug segment 114 and the fastener 110. In one or more embodiments, the suture 108 passes from the intravascular anchor 106 through the plug 112 and through the fastener 110. The fastener 110 can be moved longitudinally along the suture 108 to allow for a compressive axial force to be applied to the plug 112. For example, FIG. 1A illustrates an embodiment in which the closure device 100 is positioned across the opening 102 in the body lumen 104, but is in a non-axially compressed state. To achieve the axially compressed state of the closure device 100, the fastener 110 can be advanced along the suture 108 toward the intravascular anchor 106.

FIG. 1B provides an illustration of one embodiment of this state. As the second plug segment 116 is harder than the first plug segment 114, a sufficient compressive axial force applied through the fastener 110 as it is advanced along the suture 108 will preferentially deform the first plug segment 114 as compared to the second plug segment 116. The second plug segment 116 also resists being torn, deformed, split and/or punctured (e.g., the plug 112 migrating past the fastener 110) by the fastener 110 due to its relative hardness and/or strength. FIG. 1C provides an illustration of this axially compressed state, where the first plug segment 114 has preferentially deformed relative the deformation in the second plug segment 116.

For one or more embodiments, the fastener 110 can be advanced along the suture 108 to compress the plug 112. The fastener 110 can hold the plug 112 in the axially compressed state using a variety of techniques. One technique includes holding the plug 112 in an axially compressed state by a friction fit between the fastener 110 and the suture 108. For one or more embodiments, the cross sectional area of the suture 108 and the cross sectional area of the opening through the fastener 110 can be sufficiently different to provide for a friction fit that will reduce the chances that the fastener 110 will allow the plug to de-compress from an axially compressed state (e.g., FIG. 1C). In one or more embodiments, the suture 108 and the fastener 110 can be configured to include a one-way mechanism. The one-way mechanism can prevent the fastener 110 from reversibly moving along the suture once the fastener 110 has passed the one-way mechanism. One-way mechanisms can include, but are not limited to, a ratchet system, barbs, and combinations thereof In an additional embodiment, a thermal cutter can be used to cut the suture 108, where heat from the thermal cutter creates a ball or mass of the suture material along the suture that has a circumference greater than the remaining portions of the suture (taken longitudinally) just proximal to the fastener 110, thereby preventing the fastener 110 from reversibly moving. Other one-way mechanisms can be implemented such that the fastener 110 does not reversibly move along the suture 108 once the fastener 110 compresses the plug 112.

In one or more embodiments, the hardness of the second plug segment 116 allows for the compressive axial forces imparted through the fastener 110, as it is advanced along the suture 108, to be more equally distributed over a larger surface area onto the first plug segment 114, as compared to use of just fastener 110. Distributing the compressive axial forces over this larger surface area can minimize the chances that the second plug segment 116 and/or the fastener 110 can tear, split and/or puncture the first plug segment 114. In addition, the second plug segment 116 can also resist extrusion past the fastener 110 during hydrated expansion, which a direct result of its increased hardness and/or strength. In one or more embodiments, the compressive axial force can both axially collapse and radially expand the first plug segment 114 so as to better fill and compress against the arteriotomy.

In one or more embodiments, the first and second plug segments 114, 116 can also have different relative volumes and/or sizes. For example, as illustrated in FIG. 1 the second plug segment 116 is smaller in volume than the first plug segment 114. As discussed herein, the volume of the first plug segment 114 needs to be sufficiently large to allow for its axially collapse and radially expansion to fill and compress against the arteriotomy. The volume of the second plug segment 116, however, need not be as large as, among others, its function is to serve as a barrier to expansion past the fastener 110 during hydration and distribute the compressive axial force from the fastener 110 through to the first plug segment 114. While the second plug segment 116 may undergo some compression and/or radial expansion, it may not be to the same extent as the first plug segment 114.

In one or more embodiments, the first and second plug segments 114, 116 can be configured to provide what is referred to as “buckle control” of the plug 112. As used herein, buckle control refers to the manner in which, and the transformation in shape, that the plug 112 axially collapses and radially expands as the compressive axial force is applied through the fastener 110. In one or more embodiments, the first plug segment 114 can have channels and/or grooves formed in the body of the first plug segment 114 that allow for the first plug segment 114 to axially collapse and radially expand toward a predetermined shape. In one or more embodiments, channels and/or grooves can also be located in the body of the first plug segment 114 to minimize stresses and/or strains that may cause tears and/or ruptures in the first plug segment 114 as it is being compressed. In one or more embodiments, the channels and/or grooves can be mechanically milled or laser cut into the first plug segment 114. Alternatively, the channels and/or grooves can be formed during a casting process used to make the first plug segment 114. Examples of such structures can be found in US Pat. App. No. 2010/0217309 to Hansen et al., which is incorporated herein by reference in its entirety.

In one or more embodiments, the channels and/or grooves can have a number of different patterns, cross-sectional shapes, depths and/or combination of these features on the plug 112. Such channels and/or grooves can be provided in an effort to achieve a desired shape for the plug 112 when in the axially compressed state (e.g., buckle control). For example, one or more of the channels and/or grooves can have a helical configuration. In an additional embodiment, one or more of the channels and/or grooves can extend in a linear and/or a serpentine pattern longitudinally along the plug 112. In an additional embodiment, one or more of the channels and/or grooves can have an annular (e.g., ring like) configuration cut into the plug 112. Combinations of these shapes for the channels and/or grooves are also possible.

In one or more embodiments, the first plug segment 114 and/or the second plug segment 116 can be further modified by shaping, notching, tapering, compressing, coating with starch powder, and other features and processing to enhance the function of the plug 112 in vascular closure. For example, the first plug segment 114 and/or the second plug segment 116 can have an at least partially conical shape. In an additional embodiment, the first plug segment 114 and/or the second plug segment 116 can have a right circular cylindrical shape, an oblique cylinder shape, a pyramidal shape, a polygonal shape and/or a spherical shape, where combinations of such shapes, including the partial conical shape, are possible.

In one or more embodiments, buckle control of the plug 112 can also be achieved by providing the first plug segment 114 with regions of different hardnesses and/or strengths. As appreciated, the hardness of the material can be influenced by cross-linking and/or the presence of additional material between the material forming the plug 112 (e.g., a composite material as will be discussed herein).

In one or more embodiments, porous gelatin, collagen and combinations thereof can be suitable materials for forming the first plug segment 114 and at least a portion of the second plug segment 116. Examples of suitable collagen include, but are not limited to, Type I collagen, Type II collagen, Type III collagen, Type IV collagen, positively charged collagen (i.e., hemostatic collagen), and combinations. Other suitable materials may also be used including, for example, clot-promoting materials as are known. In one or more embodiments, starch powder and other pro-thrombogenic compounds can be used in one or both of the first plug segment 114 and the second plug segment 116.

The Type and amounts of each collagen can influence the strength and/or the hardness of the various regions of the plug 112 (e.g., the second plug segment 116). Additionally, the use of harder, water soluble sugars (e.g., sucrose, fructose, maltose, dextrose) or bioabsorbable polymers (e.g., polyglycolic acid, poly(lactic-co-glycolic) acid, polycaprolactone, polydioxanone) can also be used in creating a region of greater hardness as compared to those regions of the plug that are not treated with those materials.

In one or more embodiments, these regions of different strengths and/or hardnesses can extend radially from or toward a common longitudinal axis of the first plug segment 114 (e.g., the first plug segment 114 becoming progressively “harder” toward a common longitudinal axis of plug 112 (e.g., along the path of the suture 108), or visa versa). In one or more embodiments, the regions of different strengths and/or hardnesses can extend longitudinally along a length of the first plug segment 114 (e.g., extend along the longitudinal axis of the suture 108). Combinations of radial and longitudinal hardness and/or strength differences are also possible in the first plug segment 114, where the choice of a radial, longitudinal and/or a combination of both can be dependent upon the buckle control that is to be achieved.

In one or more embodiments, providing the first plug segment 114 with regions of different strengths and/or hardnesses can also include providing a predefined strength and/or hardness pattern to the first plug segment 114. For example, a predefined middle region extending radially across the first plug segment 114 can be provided with a strength and/or a hardness that is less than a strength and/or a hardness of either a predefined first end region and/or a predefined second end region of the first plug segment 114. In one or more embodiments, uniform and/or non-uniform strength and/or hardness gradients can also be used either longitudinally and/or radially with the first plug segment 114 to achieve a desired buckle control of the plug 112. Examples of uniform strength and/or hardness gradients can include, but are not limited to, a progressive increase or decrease in strength and/or hardness as the first plug segment 114 extends away from the second plug segment 116 toward the intravascular anchor 106. Other examples include, but are not limited to, a progressive decrease followed by a progressive increase in strength and/or hardness as the first plug segment 114 extends away from the second plug segment 116 toward the intravascular anchor 106. Other predefined hardness and/or strength patterns are also possible, where the selection thereof can be dependent upon the buckle control that is to be achieved.

In one or more embodiments, forming the second plug segment 116 can include modifying the chemical and/or physical structure of the first plug segment 114 to provide the various regions of different strengths and/or hardnesses as described herein. For example, the second plug segment 116 can be formed as a result of intentional processing steps and/or conditions that occur while forming the “bulk” material from which the plug 112 is derived. Specifically, a mass (e.g., a block or a cube) of collagen can be formed, where a layer extending into the mass from its outer surface has a strength and/or a hardness that is greater than the remaining bulk of the mass. This layer can be used to provide the second plug segment 116, while the remaining bulk provides the first plug segment 114, as the plug 112 is cut (e.g., is cored) from the mass.

In one or more embodiments, it is also possible to use a secondary process to modify the material forming the first plug segment 114 so as to form the second plug segment 16. For example, such secondary processes can include the use of heat, compression, chemical agents, cross-linking agents, and/or boding agents after initially forming a mass and/or other structure of the material (e.g., collagen).

In one or more embodiments, the second plug segment can also be formed as a composite of constituent materials that include a portion of the first plug segment and a matrix material that surrounds and supports the portion of the first plug segment 114. FIGS. 2A-2C provide an illustration of such an embodiment in which the elements provided in FIGS. 2A-2C are as discussed herein. Specifically, the second plug segment 216 can be formed as a composite of constituent materials that include a portion of the first plug segment 214 and a matrix material 220 that surrounds and supports the portion of the first plug segment 214. For example, incorporating the matrix material 220 into a portion of the first plug segment 214 can form the second plug segment 216, which results in an embodiment of the plug 212 according to the present disclosure.

An example of a suitable matrix material includes, but is not limited to, a saccharide selected from the group consisting of oligosaccharides, polysaccharides, heteropolysaccharides, sugar esters, starches, glycosaminoglycans, polyethers, and combinations thereof. Additional suitable matrix materials include poly(lactic-co-glycolic) acid, polyglycolic acid, polycaprolactone, polydioxanone. For example, in one embodiment the matrix material can include pectin (an example of a heteropolysaccharide) and hyaluronic acid (an example of a glycosaminglycan). In one or more embodiments, pectin as used herein can include homogalacturonans, substituted galacturonans, rhamnogalacturonans and combinations thereof. Specific examples of pectin include, but are not limited to, sugar beet pectin from Nu-Tek Products, LLC (Minnetonka, Minn.), sugar beet/apple/citrus pectin from Danisco A/S Langebrogade (Copenhagen, Denmark), apple/citrus pectin from Cargill Texturizing Solutions (Mechelen Belgium), apple/citrus pectin from CP Kelco Ved Banen (Skensved Denmark), and sugar beet/apple/citrus pectin from Herbstreith & Fox KG (Neuenbürg/Württ, Germany). Other specific examples of pectin are possible.

In one or more embodiments, examples of starches include, but are not limited to, carboxymethyl starch such as those sold under the trade designators Perclot (Starch Medical Inc. San Jose, Calif.), Primojel (Fonterra Excipients GmbH & Co. KG, Germany), Explotab (JRS PHARMA GMBH+CO., Germany), Vivastar P and Vivastar PSF (JRS PHARMA GMBH+CO. KG, Germany), and Glycolys distributed by Mutchler Inc. 20 Elm St., Harrington Park, N.J. 07640. The following are further examples of starch products that can be used, BleedArrest™ Clotting Powder (Hemostasis, LLC, St. Paul, Minn.), PerClot™ & SuperClot™ (Starch Medical, San Jose, Calif.) and Arista™ AH (Medafor, Minneapolis, Minn.).

In one or more embodiments, examples of sugar esters include, but are not limited to, sucrose OCTA acetate, α-D(+)-glucose pentaacetate, β-D-galactose pentaacetate, β-D-glucose pentaacetate, and combinations thereof. In one or more embodiments, examples of polyethers include, but are not limited to, polyalkylene glycols such as polyethylene glycol (PEG), methoxypoly(ethylene glycol) (MPEG), polypropylene glycol (PPG), poly(tetramethylene ether) glycol (PTMEG or PTMO), and combinations thereof. Additional examples of matrix materials can include, but are not limited to, poly L-lysine, polyvinylpyrrolidone (PVP), copolymers of PEG and hyaluronic acid, and combinations thereof.

In one or more embodiments, the matrix material includes pectin, where one or more of the other compounds listed herein for the matrix material can be included with the pectin in forming the composite material of the second plug segment 216. In one or more embodiments, pectin is a pro-thrombotic and may contribute complementary to the desired coagulation mechanism of the plug 212. As provided herein, the other compounds listed herein for the matrix material can be used as a plasticizer to increase the plasticity and the modulus of the matrix material formed with the pectin. In one or more embodiments, the amount of the plasticizer used with the pectin can be from 3 weight percent (wt. %) to 25 wt. % based on the composition of the matrix material. For example, the matrix material can be composed of 1 wt. % to 25 wt. % hyaluronic acid and 99 wt. % to 75 wt. % pectin based on the composition of the matrix material. In addition to acting as a plasticizer, hyaluronic acid may also act to improve the healing response, promoting cell migration and moderate the inflammatory response at the wound site. Additional compounds listed herein can also be included with this example matrix material composition to achieve 100 wt. % of the composition of the matrix material.

In one or more embodiments, the second plug segment 216 can be formed by incorporating the matrix material 220 into a predefined portion of the first plug segment 214. For example, an aqueous solution of pectin and hyaluronic acid, as discussed herein, can be impregnated into one or both longitudinally spaced ends of the plug 212. Examples of such impregnation procedures include, but are not limited to, dipping and/or spraying the aqueous solution of pectin and hyaluronic acid onto the defined areas of the plug 212, or covalently binding the pectin and hyaluronic acid onto the defined areas of the plug. In one or more embodiments, a vacuum assist could be used to help draw the aqueous solution of pectin and hyaluronic acid into the desired areas of the plug 212. The plug 212 can then be oven dried or lyophilized (e.g., remove the water) to form the second plug segment 216 as the composite structure discussed herein. As appreciated, pharmaceutical agents could also be included with the solution of the impregnation procedure to allow for them to be loaded onto the plug 212.

The examples of FIGS. 1A-2C illustrate the second plug segment 116, 216 at the end closest to the fastener 110, 210. It is appreciated, however, that there can be more than one of the second plug segments 116, 216 located on the plug 112, 212. For example, regions of increased hardness and/or strength (the second plug segment) can be provided along the surface of the plug 112, 212 that defines an opening for the suture 108, 208. The hardening and/or increase of strength of this region of the plug 112, 212 may help to prevent the suture 108, 208 from tearing and/or damaging the plug 112, 212 as the fastener 110, 210 is used to compress the closure device 100, 200. In one or more embodiments, the region of increased hardness and/or strength along the surface defining the opening for the suture 108, 208 need not be contiguous (e.g., there are regions where the increased hardness and/or strength take the form of rings and/or a helical spiral, by way of example).

In addition, the hardness and/or strength of other structures along the plug 112, 212 can also be modified as discussed herein. For example, the end of the plug 112, 212 closest to the intravascular anchor 106, 206 could also be part of the second plug segment. Likewise, at least a portion of a groove and/or channel present on the plug 112, 212 and/or the outer surface of the plug 112, 212 could also be part of the second plug segment. Combinations of these locations for the second plug segments are also possible.

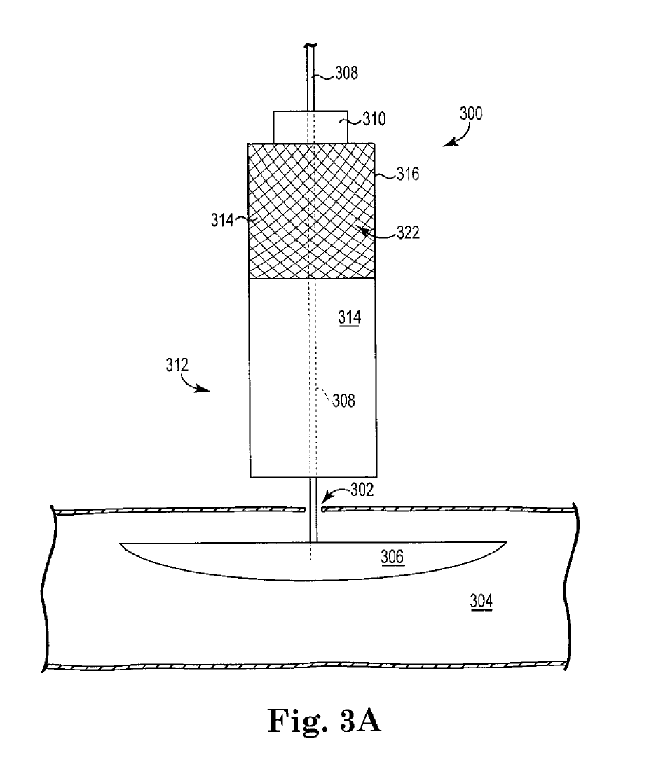

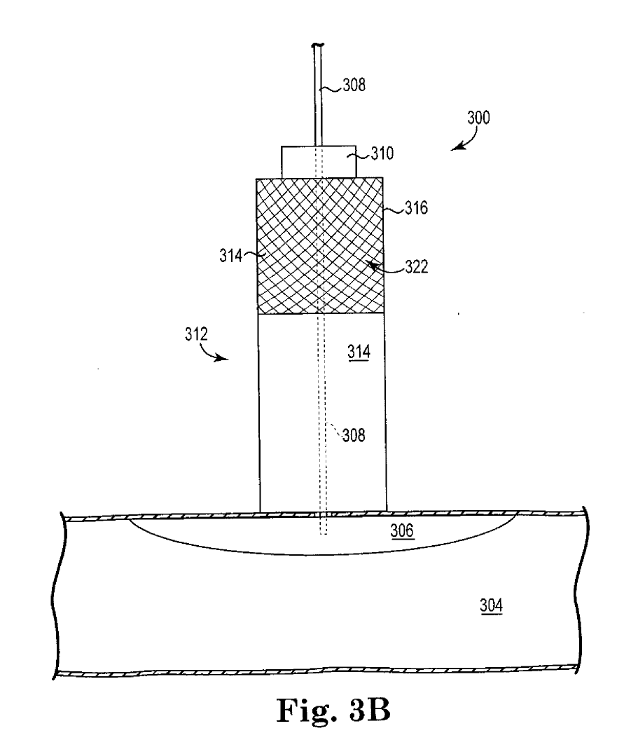

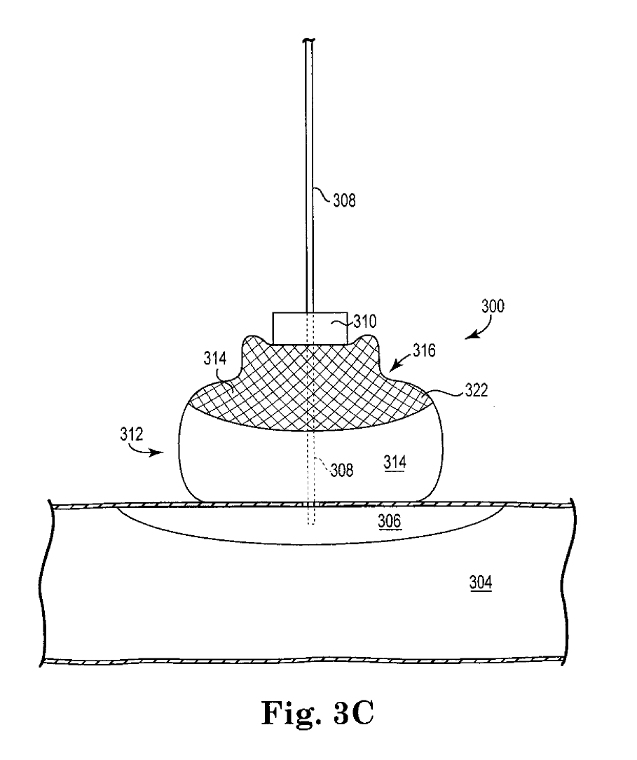

Referring now to FIGS. 3A-3C, there is illustrated an additional embodiment of the closure device 300 for closing an opening 302 in the body lumen 304 according to the present disclosure. As discussed herein, the closure device 300 includes the intravascular anchor 306 for positioning in the body lumen, the suture 308 coupled to the intravascular anchor 306, the fastener 310 joined to the suture 308 and a plug 312, where the suture 308 can pass through the opening 302 in the body lumen 304 with the intravascular anchor 306 being positioned within the lumen 304 of the vessel and the plug 312 and fastener 310 being positioned across the opening 302 from the intravascular anchor 306.

In one or more embodiments, the plug 312 has the first plug segment 314 and the second plug segment 316, where the second plug segment 316 has a hardness that is greater than the first plug segment 314. Hardness, as used herein, refers to the property of a material to resist a change of shape (e.g., permanent deformation) when a force is applied to the material. As illustrated in FIGS. 3A-3C, the first plug segment 314 is positioned between the intravascular anchor 306 and the second plug segment 316, and the second plug segment 316 is between the first plug segment 314 and the fastener 310. As discussed herein, FIG. 3A illustrates an embodiment in which the closure device 300 is positioned across the opening 302 in the body lumen 304, but is in a non-axially compressed state. To achieve the axially compressed state of the closure device 300, the fastener 310 can be advanced along the suture 308 toward the intravascular anchor 306. FIG. 3B provides an illustration of one embodiment of this state. As the second plug segment 316 is harder than the first plug segment 314, a sufficient compressive axial force applied through the fastener 310 as it is advanced along the suture 308 will preferentially deform the first plug segment 314 as compared to the second plug segment 316. The second plug segment 316 also resists being torn, deformed, split and/or punctured (e.g., the plug 312 migrating past the fastener 310) by the fastener 310 due to its relative hardness. FIG. 3C provides an illustration of this axially compressed state, where the first plug segment 314 has preferentially deformed relative the deformation in the second plug segment 316.

In one or more embodiments, the second plug segment 316 can be formed as a composite of constituent materials that include a portion of the first plug segment 314 and a mesh 322 of a reinforcement material that surrounds and supports the portion of the first plug segment 314. For example, surrounding at least a portion of the first plug segment 314 can form the second plug segment 316, which results in an embodiment of the plug 312 according to the present disclosure. In one or more embodiments, the mesh 322 can help to define a shape of the compressed second plug segment 316 (e.g., constrain its volume into a desired shape) and to distribute the compressive axial force applied by and through the fastener 310.

In one or more embodiments, the mesh 322 can have a woven, a braid, a knit and/or a felted configuration. Combinations of such configurations are also possible. The mesh 322 can be formed on the first plug segment 314 or can be formed apart from and then applied over the first plug segment 314 so as to form the second plug segment 316 having the properties discussed herein. In one or more embodiments, the mesh 322 can be formed or applied over an outer surface of the first plug segment 314 to form the second plug segment 316. In an additional embodiment, the mesh 322 can be at least partially integrated into the first plug segment 314 to form the second plug segment 316.

Examples of forming the mesh 322 on the first plug segment 314 include the use of electro-spinning techniques. In an additional embodiment, the mesh 322 can be used in conjunction with a mold used to shape at least a portion of the plug 312. The material (e.g., collagen foam) forming the plug 312 can then be cast into the mold so as to at least partially embed the mesh 322 with the plug thereby forming the second plug segment 316 discussed herein.

In one or more embodiments, the mesh 322 can have areas of different thread count of warp and filling filaments (e.g, the thread count can be the same or different in different regions of the second plug segment 316), the balance of warp and filling filaments (e.g., the number of warp filaments are different than the number of filling filaments), types of weaving (e.g., plain, basket, twill and/or satin). When knitting is used, the mesh 322 can have areas of different thread counts in terms of wales and courses used in creating the knit. Different types of knitting can include, but are not limited to warp or weft knitting (e.g., such as flat, rib, interlock, purl stitching). In addition, the spin (e.g., twist direction) of the filament (e.g., yarn or fibers) and the decitex value(s) used can also be used to modify and tailor the hardness (e.g., resistance to change of shape in response to an applied force), the hardness and/or strength and/or the buckle control of the second plug segment 316 relative the first plug segment 314.

In one or more embodiments, the mesh 322 can be formed from a bioabsorbable polymer selected from the group consisting of a homopolymer or a co-polymer of poly(glycolic acid), polylactic acid), poly(caprolactone), poly(dioxanone), poly(anhydrides), poly(ortho esters), sugars, polyketals (i.e. Poly-(1-4-phenyleneacetone dimethylene ketal (PCADK)) and combinations thereof.

In one or more embodiments, the mesh 322 closest to the fastener 310 can have a first thread count that is larger (e.g. greater hardness and/or strength) than a second thread count of the mesh 322 further from the fastener 310. So, for example, the mesh 322 that may contact the fastener 310 during the compression of the plug 312 can have a first thread count that is higher than a second thread count of the mesh that is further away from the fastener 310 (e.g, midway along the second plug segment 316). This allows for the region and/or the interface of the second plug segment 316 around the fastener 310 to be harder relative the region of the second plug segment 316 that is closer to the first plug segment 314 (e.g., the middle of the second plug segment 316).

In one or more embodiments, the mesh 322 in the non-axially compressed state of the plug 312 can freely surround (e.g., be free floating) at least a portion of the first plug segment 314 to form the second plug segment 316. It is also possible that at least a portion of the mesh 322 can be joined or bonded to the first plug segment 314 in forming the second plug segment 316. Examples of joining or bonding the mesh 322 to the first plug segment 314 in forming the second plug segment 316 can include, but are not limited to, chemical, thermal and/or mechanical bonding techniques.

In one or more embodiments, the mesh 322 can be formed in and/or applied to the first plug segment 314 that constitutes the entire size and shape of the plug 312. Alternatively, the first plug segment 314 and the second plug segment 316 can be discrete structures that are formed separately (e.g., not both derived from a common starting point of the first plug segment 314). For example, the mesh 322 can be applied to a first discrete first plug segment to form the second plug segment 316. A second discrete first plug segment 314 can then be provided on the suture 308 along with the second plug segment 316 to form the plug 312.

In one or more embodiments, the mesh 322 can surround essentially the entirety of the first discrete plug segment in forming the second plug segment 316, where different portions of the mesh 322 have different thread counts (e.g., thread densities) at different parts of the second plug segment 316 to achieve different hardnesses and/or buckle control of the second plug segment 316. For example, an end region directly adjacent (e.g., closest to) the fastener 310 and an end region directly adjacent (e.g., closest to) the first plug segment 314 can have a thread count of the mesh 322 that is higher than intermediate regions between these two end regions of the mesh 322. Other configurations of thread counts and types of construction for the mesh 322 are also possible for achieving a region of the second plug segment with a desired hardness and/or buckle control. In one or more embodiments, two or more discrete mesh 322 structures can be used in forming the second plug segment 316.

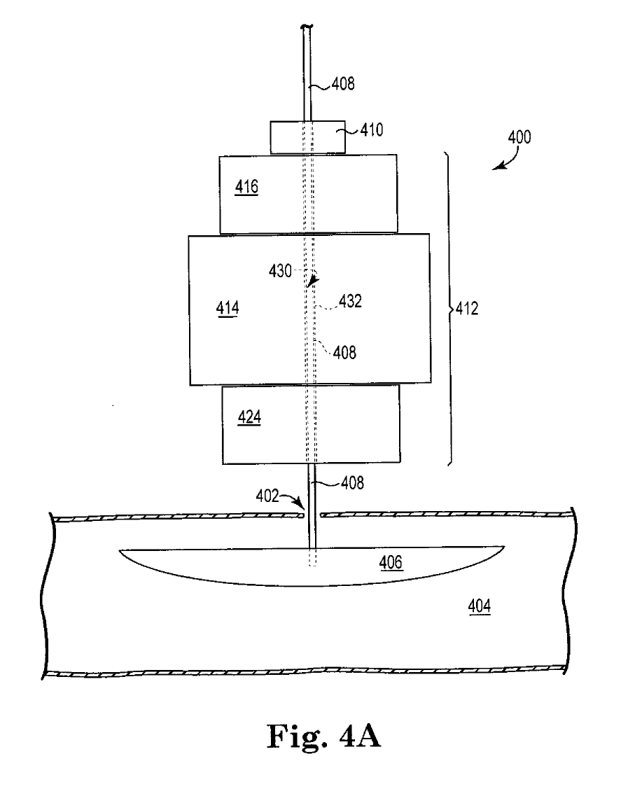

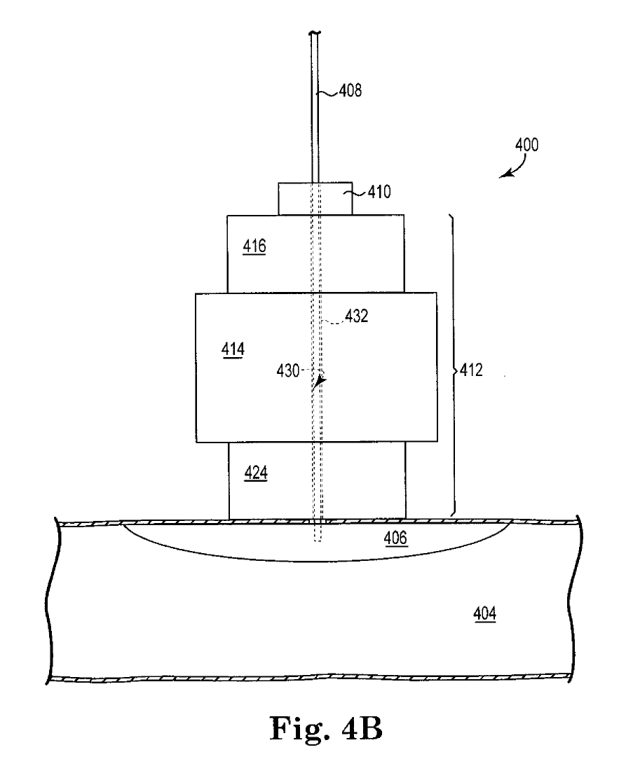

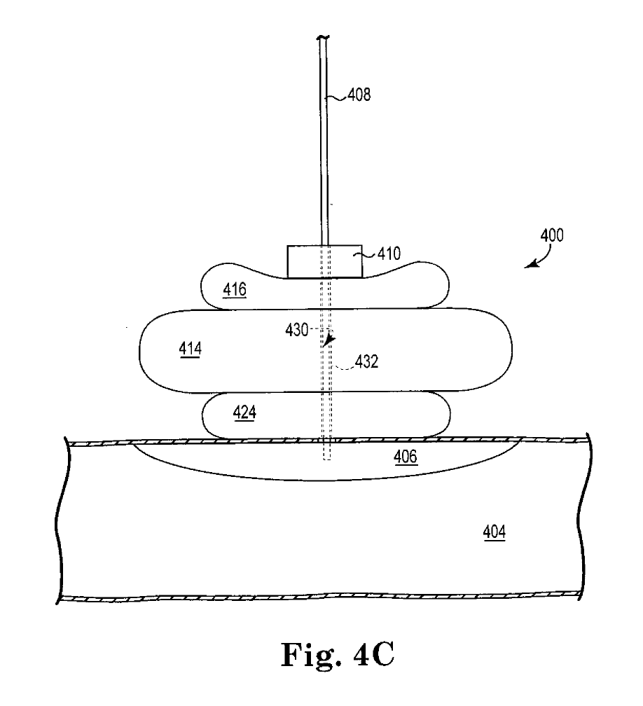

FIGS. 4A-4C provide an additional embodiment of a closure device 400 for closing an opening 402 in a body lumen 404 according to the present disclosure. As discussed herein, the closure device 400 includes the intravascular anchor 406 for positioning in the body lumen, the suture 408 coupled to the intravascular anchor 406, the fastener 410 joined to the suture 408 and an embodiment of the plug 412. In one or more embodiments, the suture 408 can pass through the opening 402 in the body lumen 404 with the intravascular anchor 406 being positioned within the lumen 404 of the vessel and the plug 412 and fastener 410 being positioned across the opening 402 from the intravascular anchor 406.

As illustrated in FIG. 4A, the plug 412 includes the first plug segment 414 and the second plug segment 416 as discrete structures oriented along the suture 408. In addition, the plug 412 includes a third plug segment 424 discrete from and oriented along the suture 408 with the first plug segment 414 and the second plug segment 416. As illustrated, the first plug segment 414 can be located between the second plug segment 416 and the third plug segment 424.

In one or more embodiments, one or more of the discrete first, second and third plug segments 414, 416 and 424 can have the same or different hardness and/or strength. For example, in one embodiment the third plug segment 424 and the second plug segment 416 each have a hardness and/or a strength that is greater than that of the first plug segment 414. In addition, the plug segments 414, 416 and 424 can each independently be formed to have the hardness and/or the strength configurations as are described herein (e.g., the composite structure of the second plug segment discussed with respect to FIGS. 2A-2C and/or the use of the mesh as discussed with respect to FIGS. 3A-3C).

In addition, the plug segments 414, 416 and 424 can each independently have one or more of the additional modifications, discussed herein, that can be used to provide for and modify buckle control of the plug 412. It is also appreciated more than three plug segments (e.g., a fourth plug segment) could also be used in forming the plug 412.

In one or more embodiments, the first plug segment 414, the second plug segment 416 and the third plug segment 424 can each have a shape that is independently selected from a cylindrical shape, a right circular cylindrical shape, an oblique cylinder shape, a pyramidal shape, a conical shape, polygonal shape and/or a spherical shape, where combinations of such shapes, including the partial conical shape, are possible.

In one or more embodiments, the relative size and/or volume of each of the first plug segment 414, the second plug segment 416 and the third plug segment 424 can be the same (e.g., each segment has a right circular cylindrical shape of approximately the same size and volume). In an additional embodiment, the relative size and/or volume of each of the first plug segment 414, the second plug segment 416 and the third plug segment 424 can be different for one or more of the segments. For example, while all three may have the same general shape, the first plug segment 414 can have a larger volume (e.g., be larger) than both the second plug segment 416 and the third plug segment 424, which themselves have approximately the same volume. Other combinations of size, volume and shape are possible.

FIGS. 4A-4C also provide an embodiment of the plug 412 in which the first plug segment 414, the second plug segment 416 and the third plug segment 424 include an interface surface 430 defining an opening 432 through which the suture 408 passes, where the interface surface 430 has a hardness and/or a strength that is greater than that of the remainder of the first plug segment 414 or other plug segments. In one embodiment, the increase in hardness and/or strength of the interface surface 430 against which the suture 408 passes can help to minimize the chances that the suture 408 can cut and/or tear into the plug segments as the compressive axial force is being applied to the fastener 410.

In one or more embodiments, the interface surface 430 defining the opening 432 can be formed by coring, laser cutting, boring and/or drilling through the material of the plug segment. The interface surface 430 can be hardened, for example, by impregnating the surface 430 with the matrix material, as discussed herein. For example, the matrix material could be sprayed and/or poured through the opening 432 to impregnate the surface, which can then be dried to form the interface surface 430 that has a hardness greater than a remainder of the first plug segment or other plug segments.

In an additional embodiment, the shape of one or more of the plug segments can be configured to better conform to the shape of the opening created in the tissue of the patient in order to form the arteriotomy. Configuring the shape of one or more of the plug segments to better conform to the shape of the opening can also help to minimize any lateral shearing forces and/or stresses imparted from the suture as the compressive axial force is being applied.

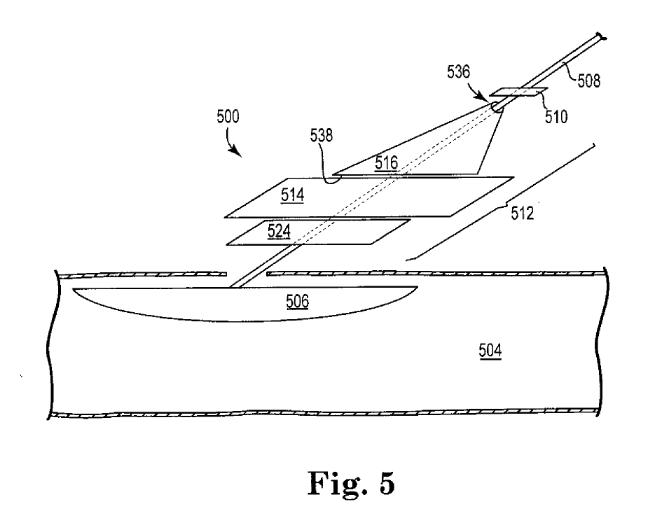

FIG. 5 provides an illustration of this embodiment in which the plug 512 includes three discrete plug segments (a first plug segment 514, a second plug segment 516, and a third plug segment 524), where the first plug segment 514 and the third plug segment 524 each have an oblique cylinder shape, and second plug segment 516 has a conical shape with an apex 536 and a base 538, where the apex 536 is between the fastener 510 and the base 538 of the conical shape. As illustrated, the compressive axial force applied through the fastener 510 remains aligned along the length of the suture 508 in part because the shape of the plug segments allows for a pressure distribution around the perimeter that is essentially equal (e.g., the plug segments do not move relative to the surface of the opening to any great degree and so the suture 508 stays centered in the plug segments). As the plug segments do not move laterally, the position of the suture 508 relative the interface surface of the segments should also remain the same.

In one or more embodiments, the suture of the present disclosure refers to a filament, fibril, or threadlike fastener. The suture may be monofilament, multifilament, or combinations thereof. The suture may be twisted, braided, or combinations thereof. For one or more embodiments, the suture may be resorbable. Resorbable, as used herein, refers to a material that can be broken down (e.g., degrade) and processed by chemical action within a human body.

For one or more embodiments, the suture is secured within the intravascular anchor with a fastening portion embedded in the intravascular anchor. In one or more embodiments, the fastening portion can be a structure formed from at least partially with the suture. In additional embodiments, other structures can be used in addition to the suture in helping to form the fastening portion. Examples of such fastening portions can be found in co-pending U.S. patent application Ser. No. ______, entitled “CLOSURE DEVICE” (BSC Dkt #10-00143 USP), which is incorporated herein in its entirety.

For one or more embodiments, the suture may degrade within a body in a period of time of 30 days to 120 days. For some applications the suture may degrade within the body in a period of time of 60 days to 90 days. For one or more embodiments, the suture may include a suture material selected from the group consisting of esters, sugars, biological materials, and combinations thereof.

For one or more embodiments, the suture may include an ester, e.g. a polyester. Examples of esters include, but are not limited to, polyglycolic acid, polylactic acid, poly(lactic-co-glycolic acid), polycaprolactone, polydioxanone, and combinations thereof.

For one or more embodiments, the suture may include a sugar. Sugar, as used herein refers to carbohydrates including monosaccharides, disaccharides, oligosaccharides, and polysaccharides having, for example, four (tetrose), five (pentose), six (hexose), seven (heptose), or more carbon atoms, and combinations thereof. Examples of monosaccharides include, but are not limited to, allose, altrose, glucose, mannose, gulose, idose, galactose, talose, ribose, arabinose, xylose, lyxose, erthrose, threose, glyceraldehyde, and combinations thereof. Examples of disaccharides include, but are not limited to, cellobiose, maltose, lactose, gentiobiose, sucrose, and combinations thereof. Examples of oligosaccharides and/or polysaccharides include, but are not limited to, cellulose, starch, amylase, amylopectin, glycogen, and combinations thereof.

For one or more embodiments, the suture may include a biological material. Examples of biological materials include, but are not limited to, surgical gut, e.g. catgut, silk, and combinations thereof. For one or more embodiments, the biological material may be treated with a chromium salt solution to provide a chromatic suture material.

For one or more embodiments, the suture may have a diameter of 0.20 millimeters to 0.700 millimeters; for example the suture may have a diameter of 0.300 millimeters to 0.500 millimeters, or 0.300 millimeters to 0.339 millimeters.

For one or more embodiments, the intravascular anchor, as disclosed herein, may be resorbable, e.g. the intravascular anchor may degrade within a body in a period of time of 30 days to 120 days. For some applications the intravascular anchor may degrade within the body in a period of time of 60 days to 90 days.

For one or more embodiments, the intravascular anchor may include an ester, as discussed herein. For one or more embodiments, the intravascular anchor may include a sugar, as discussed herein. For example, the intravascular anchor may include an intravascular anchor material selected from the group consisting of esters, sugars, and combinations thereof.

For various applications, the intravascular anchor may have differing shapes. For one or more embodiments, the intravascular anchor may include one or more polyhedron, sphere, cylinder, cone, and combinations thereof. For some applications, the intravascular anchor may have a first surface configured to appose the body lumen. For example, the first surface may be convex in relation the intravascular anchor such that the convex first surface conforms to a concave surface of the body lumen. For some applications, the intravascular anchor may have a second surface configured to help minimize flow disturbances and/or flow separation within the body lumen. For example, the second surface may be canted, e.g. where a first end and a second end of the intravascular anchor have a thickness that is less than a thickness at the center of the intravascular anchor.

For one or more embodiments, the plug, as disclosed herein, may be resorbable, e.g. the plug may degrade within a body in a period of time of 30 days to 120 days. For some applications the plug may degrade within the body in a period of time of 60 days to 90 days. In one or more embodiments, the plug can bioabsorb at different rates, as it is formed from different materials. So, for example, the collagen foam of the plug, which can be about 30 wt. % of the mass, may take about 60 days to bioabsorb, while the starch powder, which can be about 70 wt. % of the mass, can take about 2-3 days to bioabsorb. So 70 wt. % of the plug mass can be bioabsorbed in days and the remaining 30 wt. % takes may take a couple months.

For various applications the plug in the pre-deployed state may have differing dimensions. For one or more embodiments, plug in the pre-deployed state may have a length of 0.5 centimeters to 5 centimeters; for example the plug in the pre-deployed state may have a length of 0.75 centimeters to 4 centimeters, 1 centimeter to 3 centimeters, or 1.9 centimeters. For one or more embodiments, plug in the pre-deployed state may have a diameter of 0.10 centimeters to 2 centimeters; for example the plug in the pre-deployed state may have a diameter of 0.1 millimeters to 1.5 centimeters, or 0.17 centimeters to 0.3 centimeter.

The fastener, as disclosed herein, may be resorbable, e.g. the fastener may degrade within a body in a period of time of 30 days to 120 days. For some applications the fastener may degrade within the body in a period of time of 60 days to 90 days.

For one or more embodiments, the fastener may include an ester, as discussed herein. For one or more embodiments, the fastener may include a sugar, as discussed herein.

For one or more embodiments, the fastener may include an ester, as discussed herein. For one or more embodiments, the fastener may include a sugar, as discussed herein.

In one or more embodiments, the present disclosure also includes a method of making a closure device, as described herein, for closing an opening in a body lumen. In one or more embodiments, the first plug segment of a plug of the closure device and the second plug segment of the plug of the closure device can be formed, as discussed herein, where forming the second plug segment includes making the hardness of the second plug segment greater than the first plug segment, as discussed herein. The suture can then be passed through the plug with the first plug segment between an intravascular anchor and the second plug segment. The fastener can then be joined to the suture with the second plug segment between the first plug segment and the fastener.

As discussed herein, the hardness and/or strength of the second plug segment can be increased so as to be greater than the hardness and/or strength of the first plug segment. In one or more embodiments, making the hardness and/or strength of the second plug segment greater than the first plug segment can include, but is not limited to, infusing the matrix material, as discussed herein, into a portion of the first plug segment where the matrix material surrounds and supports the portion of the first plug segment to form the second plug segment. In an additional embodiment, forming the second plug segment can include joining the mesh of a reinforcement material, as discussed herein, to surround and support a portion of the first plug segment.

In one or more embodiments, joining the mesh can include forming the mesh with a first area having a first thread count and a second area having a second thread count, the first thread count being larger than the second thread count of the mesh, and positioning the first area of the mesh in the second plug segment closest to the fastener to allow the fastener to contact the first area.

In one or more embodiments, the plug segments can be formed into different shapes, as discussed herein, using a variety of techniques. These can include, but are not limited to, milling techniques, coring techniques, boring techniques, casting techniques, molding techniques, lasering techniques, to name only a few. For example, milling techniques could be used to form a block of collagen into a conical shape with an apex and a base (such as that shown in FIG. 5 for the second plug segment 516, where the apex 536 is between the fastener 510 and the base 538 of the conical shape).

In one or more embodiments, it is also possible to form the first plug segment, the second plug segment and, optionally the third plug segment, from a single mass of collagen, as discussed herein. Alternatively, each of the first plug segment and the second plug segment, and optionally the third plug segment, could be formed as discrete structures through which the suture can be passed, as provided herein. As appreciated, the third plug segment could be made to have a hardness and/or a strength that is greater than the hardness and/or strength of the first plug segment. In one or more embodiments, each of the first plug segment, the second plug segment and the third plug segments could be formed in an identical geometric shape. Alternatively, one or more of the first plug segment, the second plug segment and the third plug segment could be formed to have a different geometric shape.

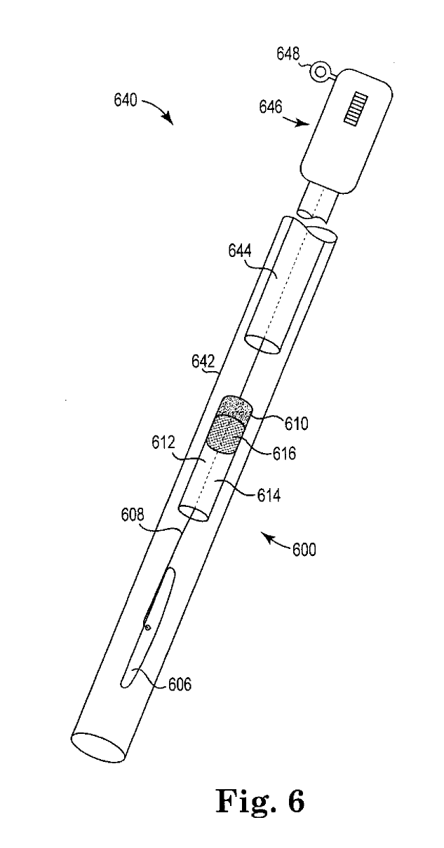

Embodiments of the present disclosure provide a system for closing an opening in a body lumen. FIG. 6 illustrates a system 640 for closing an opening in a body lumen. The system includes a sheath 642, a closure device 600 releasably housed in the sheath 642, and a push member 644 disposed in the sheath 642, where the push member 644 extends to advance the closure device 600 from the sheath 642 and to apply the compressive axial force to the fastener 610 in deploying the closure device 600.

Push member 644 is disposed in the sheath 642 and configured to advance the closure device 600 out from the sheath 642. Push member 644 may be located, for example, adjacent the closure device 600. While the push member is illustrated as a generally cylindrical elongate member, the push member may include one or more polyhedron, sphere, cylinder, cone, and combinations thereof

Closure device 600 may be releasably housed in the sheath 642. Closure device 600 may include the intravascular anchor 606 for positioning in the body lumen, the suture 608 coupled to the intravascular anchor 606, the fastener 610 joined to the suture 608 and the plug 612 having the first plug segment 614 and the second plug segment 616, as discussed herein. The intravascular anchor 606 may include a furrow and/or channel along a portion of the intravascular anchor. The furrow and/or channel may be located along a central portion of the intravascular anchor 606. The intravascular anchor 606 including the furrow and/or channel may be foldable within the sheath 642.

For one or more embodiments, the system 640 may include a handle 646. Handle 646 may include one or more control members, such as but not limited to, a slider 648. The one or more control members may be coupled to the intravascular anchor 606 and may help control positioning of the intravascular anchor 606. Handle 646 may also include a number of different and/or alternative structural features.

For one or more embodiments, the system 640 may include one or more actuation members 648. The one or more actuation members 648 may be coupled to the sheath 642 and/or the suture 608. The one or more actuation members 648 may function to retract the sheath 642, to apply a tension force to the suture 608 and/or apply the compressive axial force to the fastener 610 in deploying the closure device 600. Once deployed, the sheath 642 can be removed and suture 608 not being used to compress the closure device 600 can be cut and removed.

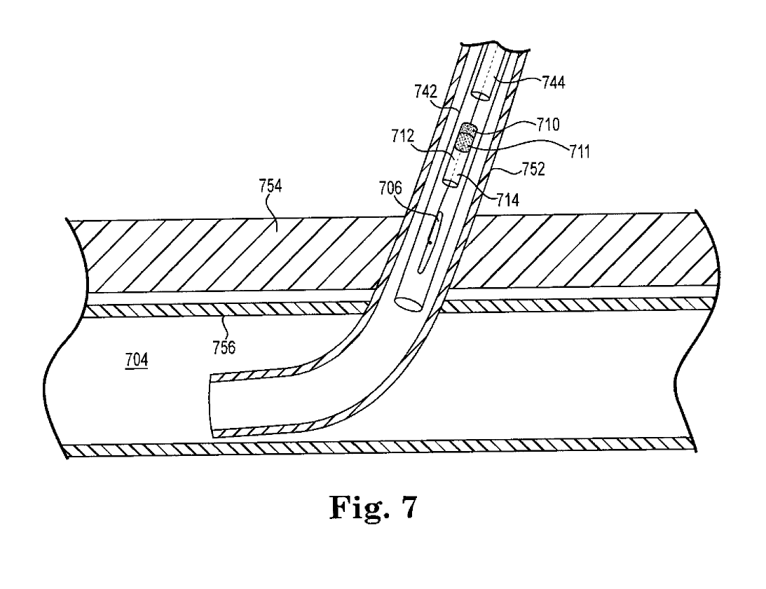

FIG. 7 illustrates the system disposed within an introducer sheath 752. As illustrated in FIG. 7, the introducer sheath extends through the skin 754 and into the body lumen 704, e.g. the femoral artery. While FIG. 7 illustrates that for one or more embodiments the system may be utilized with the introducer sheath 752, it is contemplated that the system may be deployed without an introducer sheath, e.g. for applications where an introducer sheath has previously been removed. Deployment of the system may include the use of an obturator and/or dilator.

For some applications, the system may be advanced through the introducer sheath 752 to a position where the closure device may be advanced out from the introducer sheath 752 and into the body lumen 704.

After and/or while being advanced out from the introducer sheath 752, the intravascular anchor 706 may be configured to shift and/or tilt to prepare for engagement with body lumen wall 756. The shifting and/or tilting may be accomplished in a number of different ways. For example, suture 708 may be configured or otherwise be arranged in conjunction with the intravascular anchor 706 so that the suture 708 may be manipulated to cause the intravascular anchor 706 to shift and/or tilt. For some applications, the suture may be wrapped and/or wound around one or more portions of the intravascular anchor.

For some applications, when the intravascular anchor 708 is prepared for engagement with body lumen wall 756, sheath 742 and/or introducer sheath 752 may be withdrawn, e.g. moved proximally, from the body lumen 704 so that the intravascular anchor 706 is positioned in a desired location, such as engaging or imminently prepared to engage the body lumen wall 756. After and/or while the intravascular anchor 706 is positioned in the desired location the push member 744 may be advanced, e.g. moved distally, so as to engage and apply the compressive axial force to the fastener 710 in deploying the closure device 700, as discussed herein. For one or more embodiments, the push force applied to the fastener 710 via the push member is sufficient to deform the plug 712, e.g. transition the plug from the pre-deployed state to the deformed state. For one or more embodiments, the deformed plug engages an abluminal surface of the body lumen and/or a portion of the intravascular anchor.

As discussed herein, a tension force may be applied to the suture 708. The tension force may be applied prior to, concurrently with, and/or after the compressive axial force is applied to the plug fastener 710 and the plug 712. Application of the tension force may pull together and/or secure the intravascular anchor 706 with the plug 712. For one or more embodiments, during application of the tension force the suture 708 does not move relative to the intravascular anchor 706. Once desirably situated, e.g. the intravascular anchor 706 engaging the body lumen wall and the deformed plug 712 engaging the abluminal surface of the body lumen and/or a portion of the intravascular anchor, the sheath 742 and/or the introducer sheath 752 may be retracted to leave the closure device closing 700 the opening in the body lumen. Excess suture, e.g. a portion of suture extending from the desirably situated closure device, may be removed.

It is to be understood that the above description has been made in an illustrative fashion, and not a restrictive one.

We claim:

1. A closure device for closing an opening in a body lumen comprising:
an intravascular anchor for positioning in the body lumen;
a suture coupled to the intravascular anchor, the suture to pass through the opening in the body lumen;
a fastener joined to the suture, where the fastener moves longitudinally along the suture; and
a plug having a first plug segment and a second plug segment, the second plug segment having a hardness greater than the first plug segment, where the suture passes through the plug with the first plug segment between the intravascular anchor and the second plug segment and the second plug segment between the first plug segment and the fastener.

2. The closure device of claim 1, where the second plug segment is a composite of constituent materials that include a portion of the first plug segment and a matrix material that surrounds and supports the portion of the first plug segment.

3. The closure device of claim 2, where the matrix material includes a saccharide selected from the group consisting of oligosaccharides, polysaccharides, heteropolysaccharides, sugar esters, starches, glycosaminoglycans, polyethers, and combinations thereof.

4. The closure device of claim 2, where the matrix material includes pectin and hyaluronic acid.

5. The closure device of claim 1, where the second plug segment has a conical shape with an apex and a base, where the apex is between the fastener and the base of the conical shape.

6. The closure device of claim 1, where the second plug segment is a composite of constituent materials that include a portion of the first plug segment and a mesh of a reinforcement material that surrounds and supports the portion of the first plug segment.

7. The closure device of claim 6, where the mesh closest to the fastener has a first thread count that is larger than a second thread count of the mesh further from the fastener.

8. The closure device of claim 6, where the mesh is formed from a bioabsorbable polymer selected from the group consisting of poly(glycolic acid), poly(lactic acid), poly(caprolactone), poly(dioxanone), poly(anhydrides), poly(ortho esters), polyketals and combinations thereof

9. The closure device of claim 1, where the second plug segment has a strength that is greater than the first plug segment.

10. The closure device of claim 9, where the first plug segment and the second plug segment are discrete structures oriented along the suture.

11. The closure device of claim 10, where the plug includes a third plug segment discrete from and oriented along the suture with the first plug segment and the second plug segment, where the first plug segment is located between the second plug segment and the third plug segment.

12. The closure device of claim 11, where the third plug segment and the second plug segment each have a strength that is greater than the first plug segment.

13. The closure device of claim 11, where the first plug segment, the second plug segment and the third plug segments are cylindrical.

14. The closure device of claim 1, where first plug segment and the second plug segment include an interface surface defining an opening through which the suture passes, the interface surface having a hardness greater than a remainder of the first plug segment.

15. A system for closing an opening in a body lumen comprising:
a sheath;
a closure device releasably housed in the sheath, the closure device including:
an intravascular anchor for positioning in the body lumen;
a suture coupled to the intravascular anchor, the suture to pass through the opening in the body lumen;
a fastener joined to the suture, where the fastener moves longitudinally along the suture; and
a plug having a first plug segment and a second plug segment, the second plug segment having a hardness greater than the first plug segment, where the suture passes through the plug with the first plug segment between the intravascular anchor and the second plug segment and the second plug segment between the first plug segment and the fastener; and
a push member disposed in the sheath, where the push member extends to advance the closure device from the sheath.

16. The system of claim 15, where the second plug segment is a composite of constituent materials that include a portion of the first plug segment and a matrix material that surrounds and supports the portion of the first plug segment.

17. The system of claim 15, where the second plug segment has a conical shape with an apex and a base, where the apex is between the fastener and the base of the conical shape.

18. The system of claim 15, where the second plug segment is a composite of constituent materials that include a portion of the first plug segment and a mesh of a reinforcement material that surrounds and supports the portion of the first plug segment.

19. The system of claim 15, where the first plug segment and the second plug segment are discrete structures oriented along the suture.

20. The system of claim 19, where the plug includes a third plug segment discrete from and oriented along the suture with the first plug segment and the second plug segment, where the first plug segment is located between the second plug segment and the third plug segment.
